(19) United States
(12) Patent Application Publication
(10) Pub. No.: US 2021/0401494 A1
(43) Pub. Date: Dec. 30, 2021

(54) PULMONARY ARTERIAL HYPERTENSION CATHETERS

(71) Applicant: NXT Biomedical, LLC, Irvine, CA (US)

(72) Inventors: Joseph Passman, Irvine, CA (US); Alexander Siegel, Irvine, CA (US); Glen Rabito, Irvine, CA (US); Stanton J. Rowe, Irvine, CA (US); Elliot Howard, Irvine, CA (US); Abubaker Khalifa, Irvine, CA (US); Robert C. Taft, Irvine, CA (US); Linda Thai, Irvine, CA (US)

(73) Assignee: NXT Biomedical, LLC, Irvine, CA (US)

(21) Appl. No.: 17/357,830

(22) Filed: Jun. 24, 2021

Related U.S. Application Data
(60) Provisional application No. 63/043,645, filed on Jun. 24, 2020.

(51) Int. Cl. A61B 18/14

(52) U.S. Cl. CPC A61B 18/1492; A61B 2018/00351; A61B 2018/141

(57) ABSTRACT

Disclosed herein are devices and methods for creating a shunt between two vessels or lumens within a patient. While these devices and methods are generally described with regard to treatment of hypertension (e.g., pulmonary arterial hypertension) and/or right heart failure/disfunction, they can be used with a variety of different vessels and lumens for other purposes. The devices include puncturing guidewire embodiments that can more accurately pierce two vessels, as well as snare catheter designs that can prevent unwanted damage from a puncturing guidewire.

### RELATED APPLICATIONS

This application claims benefit of and priority to U.S. Provisional Application Ser. No. 63/043,645 filed Jun. 24, 2020 entitled Pulmonary Arterial Hypertension Catheters, which is hereby incorporated herein by reference in its entirety.

### BACKGROUND OF THE INVENTION

Pulmonary hypertension is a condition that describes high blood pressure in the lungs. There are a variety of causes for the increased pulmonary blood pressure, including obstruction of the small arteries in the lung, high left-sided heart pressures, and chronic lung disease.

There are many medical conditions that also create high pulmonary blood pressure as a secondary condition, including heart failure. In heart failure, the heart is unable to meet the demand for blood coming from the body. This often leads to increased pressures within the heart that can back up into the lungs causing pulmonary hypertension at rest or during exercise.

In almost all cases, this increased pulmonary blood pressure causes the right ventricle to work harder to supply the lungs and the left side of the heart with blood. Over time, this additional load causes damage to the heart, decreasing efficiency and limiting the ability to keep up with the demands of the body, especially during exercise.

Reducing pulmonary blood pressure has been the target of numerous therapies, especially in patients with pulmonary arterial hypertension where several drugs have shown moderate success. However, these drugs are often very expensive and burdensome to the patient and over time can lose their effectiveness.

In this regard, what is needed is an improved treatment option for reducing pulmonary blood pressure and other conditions of elevated blood pressure.

### SUMMARY OF THE INVENTION

Disclosed herein are improved devices and methods for creating a shunt between two vessels or lumens within a patient. While the devices and methods may be particularly useful in creating a shunt between a superior vena cava (SVC) and a right pulmonary artery (RPA), other shunt locations are also possible.

One embodiment is directed to a delivery device catheter configured to deliver a shunt support structure without a sheath over the delivery device while crossing one or more vessel walls. The catheter can include one or more proximal or distal cones that cover only a proximal and/or distal end of the support structure. The cones can be slidable and biased to a position covering the support structure or can be configured to at least partially rip or tear away.

Another embodiment is directed to a delivery device with a distal tip having one or more RF electrodes configured such that the delivery device can pierce one or more vessel walls, dilate one or more vessel walls, and then deliver a shunt support structure to create a shunt between two vessels.

Another embodiment is directed to a radiofrequency piercing guidewire having a biased outer sheath. The outer sheath covers the distal tip of the guidewire in one position and then slides back a predetermined distance to a second position to expose the ablative tip of the guidewire. This may limit the length that the guidewire tip can penetrate beyond a wall of a vessel.

Yet another embodiment is directed to a handle for a radiofrequency piercing guidewire that includes a mechanism (e.g., a thumbwheel or screw drive mechanism) to advance the guidewire out of a sheath a predetermined distance to thereby prevent overextension completely through a vessel wall.

Another embodiment is directed to a snare catheter having an inflatable balloon at its distal tip with one or more snare loops positioned within the balloon, outside of the balloon, or embedded in the balloon material.

Yet another embodiment is directed to a snare catheter having a shield disposed on one side of one or more snare loops. The shield is configured to prevent a piercing guidewire from extending through it.

Another embodiment is directed to a snare catheter having one or more balloons and a shield member. The one or more balloons can be configured to anchor the distal end of the catheter and or center or brace the distal end of the catheter in a desired position. The one or more balloons can be located proximally and/or distally of the shield member.

Yet another embodiment is directed to a snare catheter having one or more perfusion passages. The one or more perfusion passages may extend through one or more balloons or may extend through a body of the catheter.

Another embodiment is directed to a snare catheter with a radiofrequency electrode to help direct radiofrequency current form an RF puncturing guidewire.

Yet another embodiment is directed to a snare catheter having a conductive coil configured to generate a magnetic field. The magnetic field can be used by a puncturing guidewire to sense a position of the conductive coil of the snare and/or to magnetically attract the puncturing guidewire via magnetic force.

Another embodiment is directed to a steerable catheter that includes one or more balloons or expandable rings for positioning and/or bracing a distal end of the catheter. This may allow a puncturing guidewire to more accurately be deployed from the steerable catheter.

Yet another embodiment includes one or more combinations of any of the features of the embodiments of this specification, as well as one or more combinations of methods of use of any of the embodiments of this specification.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a view of a heart with a diagnostic catheter.

FIG. 2 is a view of a heart with a snare catheter.

FIG. 3 is a view of vessel with a snare catheter within it.

FIG. 4 is a view of a heart with a snare catheter and a puncture system.

FIG. 5 is a view of a vessel with a snare catheter and a puncture system.

FIG. 6 is a view of a vessel with a snare catheter and a puncture system.

FIG. 7 is a view of a vessel with a snare catheter and a puncture system.

FIG. 8 is a view of a vessel with a snare catheter and a puncture system.

FIG. 9 is a view of a shunt support structure forming a stent between two vessels.

FIG. 10 is a view of a compressed shunt support structure.

FIG. 11 is a view of an expanded shunt support structure.

FIG. 12 is a view of a compressed shunt support structure.

FIG. 13 is a view of an expanded shunt support structure.

FIG. 14 is a view of a shunt support structure delivery catheter.

FIG. 15 is a view of a shunt support structure delivery catheter.

FIG. 16 is a view of a shunt support structure delivery catheter.

FIG. 17 is a view of a shunt support structure delivery catheter.

FIGS. 18, 19, and 20 are views of a puncturing guidewire.

FIGS. 21 and 22 are views of a puncturing guidewire handle.

FIG. 23 is a view of a snare catheter.

FIG. 24 is a view of a snare catheter.

FIG. 25 is a view of a snare catheter.

FIG. 26 is a view of a snare catheter and a puncturing guidewire.

FIG. 27 is a view of a snare catheter and a puncturing guidewire.

FIG. 28 is a view of a snare catheter and a puncturing guidewire.

FIG. 29 is a view of a snare catheter and a puncturing guidewire.

FIG. 30 is a view of a snare catheter and a puncturing guidewire.

FIG. 31 is a view of a snare catheter and a puncturing guidewire.

FIG. 32 is a view of a snare catheter and a puncturing guidewire.

FIG. 33 is a view of a steerable or crossing catheter.

FIG. 34 is a view of a steerable or crossing catheter.

FIG. 35 is a view of a steerable or crossing catheter.

FIG. 36 is a view of a steerable or crossing catheter.

FIG. 37 is a view of steerable or crossing catheter.

FIG. 38 is a view of steerable or crossing catheter.

FIG. 39 is a view of catheter with a side aperture.

FIG. 40 is a view of catheter with a side aperture.

FIG. 41 is a view of catheter with a side aperture.

FIG. 42 is a view of catheter with a side aperture.

FIG. 43 is a view of catheter with a side aperture.

FIG. 44 is a view of catheter with a side aperture.

FIGS. 45, 46, 47, 48, and 49 are views of a catheter with a side aperture and radiopaque markers.

FIGS. 50, 51, 52, and 53 are views of a catheter with a side aperture and a magnetic connection mechanism.

FIG. 54 is a view of two catheters with a magnetic connection mechanism.

FIGS. 55, 56, and 57 are views of a balloon snare catheter.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements. While different embodiments are described, features of each embodiment can be used interchangeably with other described embodiments. In other words, any of the features of each of the embodiments can be mixed and matched with each other, and embodiments should not necessarily be rigidly interpreted to only include the features shown or described.

Disclosed herein are improved devices and methods for creating a shunt between two vessels or lumens within a patient. While these devices and methods are generally described with regard to treatment of hypertension (e.g., pulmonary arterial hypertension) and/or right heart failure/disfunction, it should be understood that they can be used with a variety of different vessels and lumens for other purposes.

Shunts can be used to connect several different locations within a body for treatment of pulmonary arterial hypertension and/or right heart failure/disfunction. This specification will primarily discuss embodiments of the present invention regarding a shunt connecting a right pulmonary artery (RPA) to a superior vena cava (SVC). However, these embodiments should not be limited to use solely at this location, as use with shunts at other locations is specifically contemplated.

A general example procedure for creating a shunt between a right pulmonary artery and a superior vena cava will be discussed below and further modifications of this procedure and its equipment will then be discussed. In this respect, it is intended that the different embodiments discussed in this specification can be mixed and matched in any combination, particularly with shunt procedure described between a right pulmonary artery and a superior vena cava.

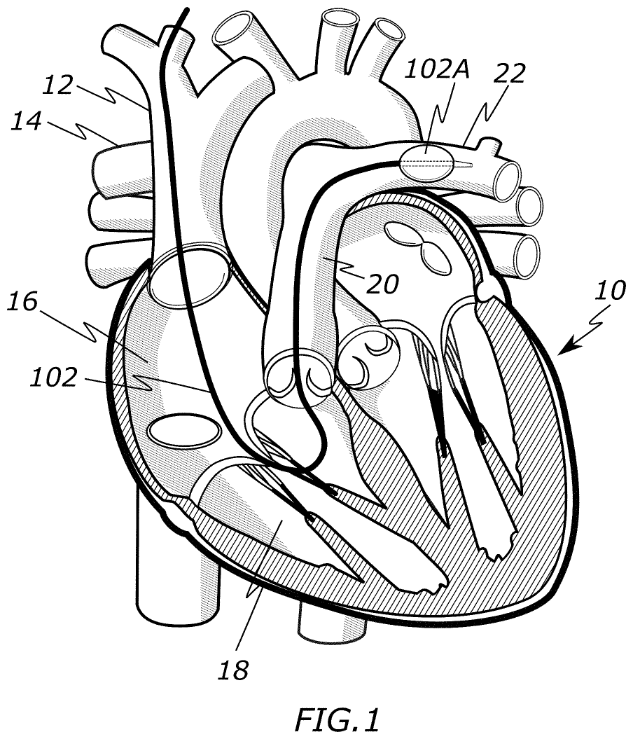

FIGS. 1-12 illustrate various aspects of a method and equipment for creating a shunt 30 between a right pulmonary artery 14 and a superior vena cava 12. While the crossing is performed from the superior vena cava 12 into the right pulmonary artery 14, the opposite may also be performed, crossing from the right pulmonary artery 14 into the superior vena cava 12. The resulting shunt connection may decrease the total pulmonary vascular resistance and the afterload of the right ventricle. The method generally includes the steps of targeting the right pulmonary artery 14, crossing through the superior vena cava 12 to the right pulmonary artery 14 (or vice versa), positioning a shunt support structure 120 between both vessels 12, 14, and removing the delivery system to establish the shunt 30.

Optionally, pre-implant hemodynamic or blood flow-related data may first be acquired from the patient to determine or characterize any abnormalities exist in the heart and lungs. For example, a Swan-Ganz catheterization procedure can be performed, as seen in FIG. 1, to allow pressure measurement in the right atrium, pulmonary artery, and pulmonary capillaries. The pulmonary artery catheter 102 is typically advanced into the right atrium 16 and a balloon tip 102A is inflated, allowing the balloon tip 102A to be carried into the right ventricle 18, into the pulmonary trunk 20, and into the left pulmonary artery 22, which lead to the lungs.

Next, a target and/or grasping device is placed in one of the vessels followed by a piercing device can be placed in the other vessel, allowing one device to pierce both vessels and the other device to grab or engage the piercing device. For example, the target and/or grasping device can be positioned within the right pulmonary artery 14 and the piercing device can be placed in the superior vena cava 12, or vice versa.

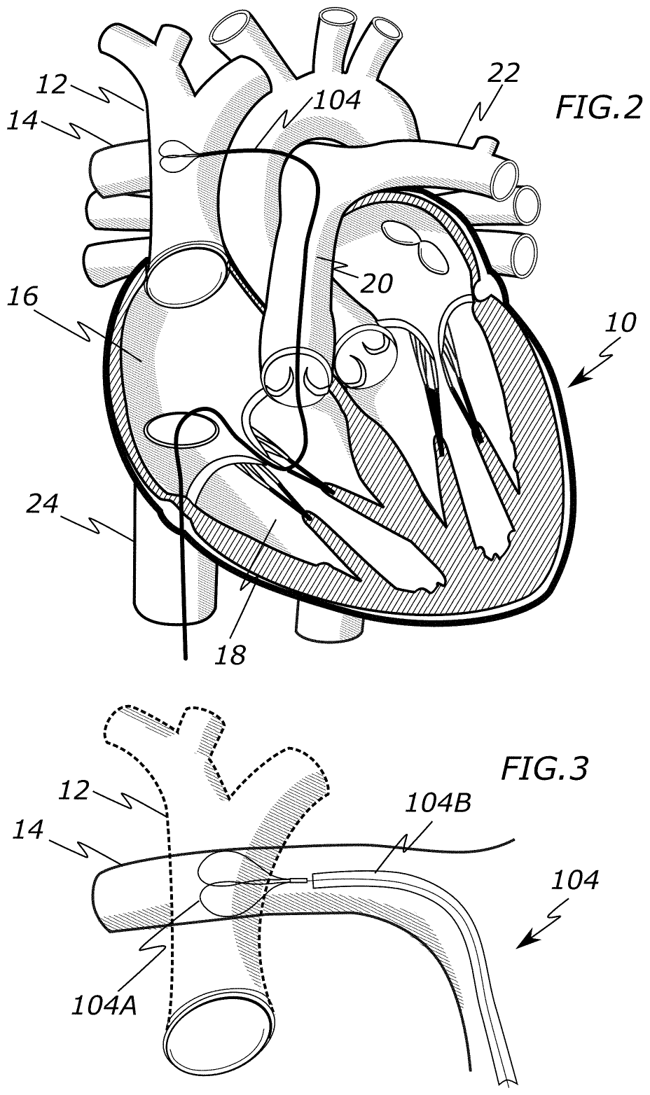

FIGS. 2 and 3 illustrate placing a target and/or grasping device in the right pulmonary artery 14. In this example, the target and/or grasping device is a snare catheter 104 that includes one or more loops 104A that can be retracted into an outer sheath 104B. Additionally, radiopaque markers may be included on the catheter 104 to help “target” the loops 104A of the snare catheter. The loops 104A can be composed of a wire, such as a metal or polymer wire.

The snare catheter 104 can be placed, in one example, through the inferior vena cava 24, into the pulmonary trunk 20, and further into the right pulmonary artery 14. Placement can be achieved with a variety of techniques, including via floating an arrow balloon catheter to the desired location and then advancing the snare catheter 104 within the arrow catheter or other catheter or guidewire advanced to the location of the arrow catheter.

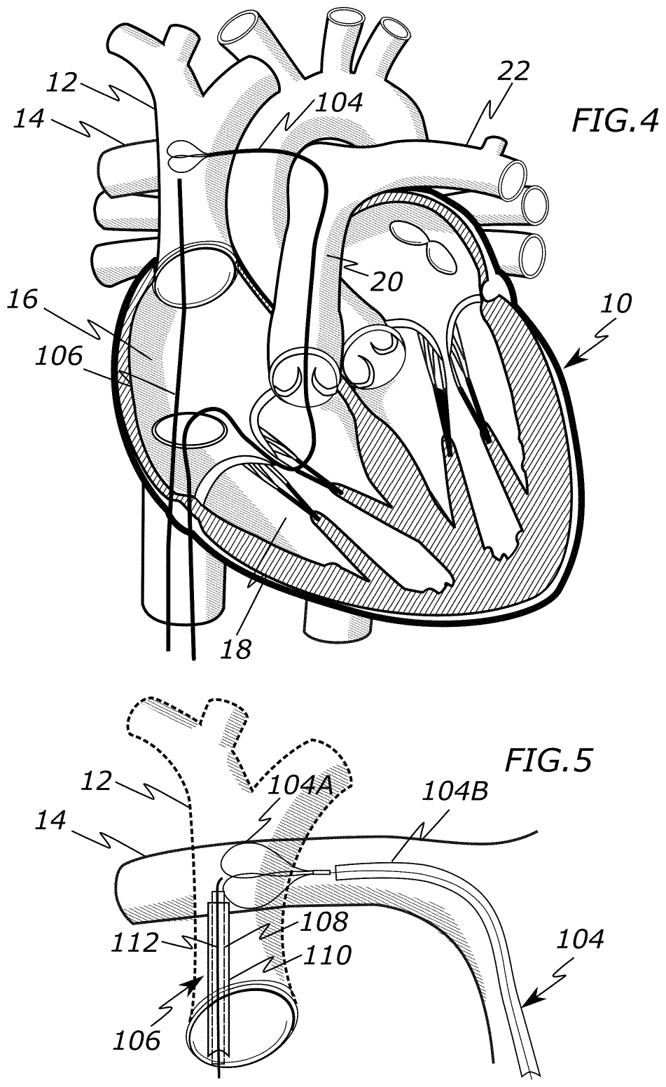

FIGS. 4 and 5 illustrate introducing the puncture system 106 in the superior vena cava 12. In one example, the puncture system 106 may include an outer steerable catheter 110 (e.g., an Agilis catheter), a flexible crossing catheter 108 positioned within the outer steerable catheter 110, and a puncturing guidewire 112 (e.g., an RF guidewire) positioned within the crossing catheter 108. However, other puncturing systems are possible. The puncture system 106 may be introduced by first accessing the femoral vein with a 12 Fr catheter sheath. Next, a guidewire (e.g., 0.035″) is advanced to the superior vena cava 12. An outer steerable catheter 110 (Agilis catheter) is tracked over the guidewire into the superior vena cava 12. Finally, the guidewire is exchanged for the crossing catheter 108 and the inner puncturing guidewire 112.

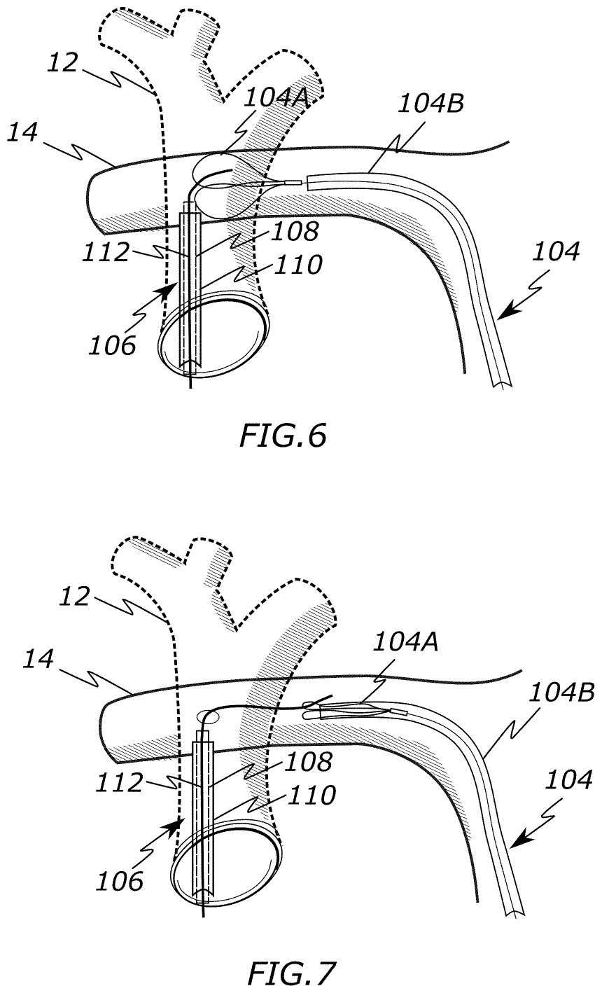

FIGS. 6 and 7 illustrate the process of puncturing the superior vena cava 12 and the right pulmonary artery 14. The tip of the crossing catheter 108 can be angled or directed towards the desired puncture location (e.g., by directing or adjusting the position of the steerable catheter 110) and the tip angle and position can be confirmed via fluoroscopy. Next, the puncturing guidewire 112 is advanced into the target location via its puncturing method (e.g., by activating radiofrequency energy) so that it passes through the wall of the superior vena cava 12, through the wall of the right pulmonary artery 10, and into one of the loops 104A of the snare catheter. The position of the puncturing guidewire 112 within one of the loops 104A can be confirmed via imaging techniques. As seen in FIG. 7, the loops 104 of the snare 104 can be at least partially withdrawn into the outer sheath 1046 to grab or capture the puncturing guidewire 112. It also may be necessary to advance the crossing catheter 108 or a different dilator catheter through the punctures to dilate the openings. In that respect, the crossing catheter 108 preferably has a dilating tip.

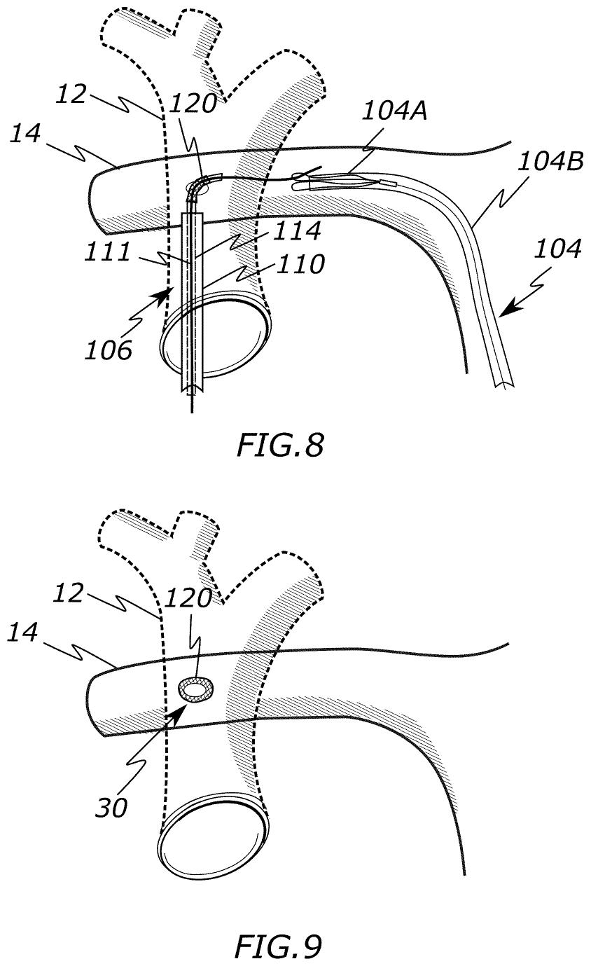

Next, as seen in FIG. 8, a shunt support structure 120 is delivered between the superior vena cava 12 and the right pulmonary artery 14. For example, the puncturing guidewire 112 may be exchanged for a delivery guidewire 111 and the crossing catheter 108 can be removed, allowing a delivery catheter 114 to be advanced over the delivery guidewire 111. The distal end of the delivery catheter 114 is positioned between the superior vena cava 12 and the right pulmonary artery 14. The delivery catheter 114 can have an outer sheath that is withdrawn to expose the shunt support structure 120 and the shunt support structure 120 can be radially expandable by either self-expanding, by a balloon being inflated within the structure 120, or a combination of both methods. The support structure 120 may include a passage therethrough, which creates the shunt passage 30 between both vessels 12 and 14.

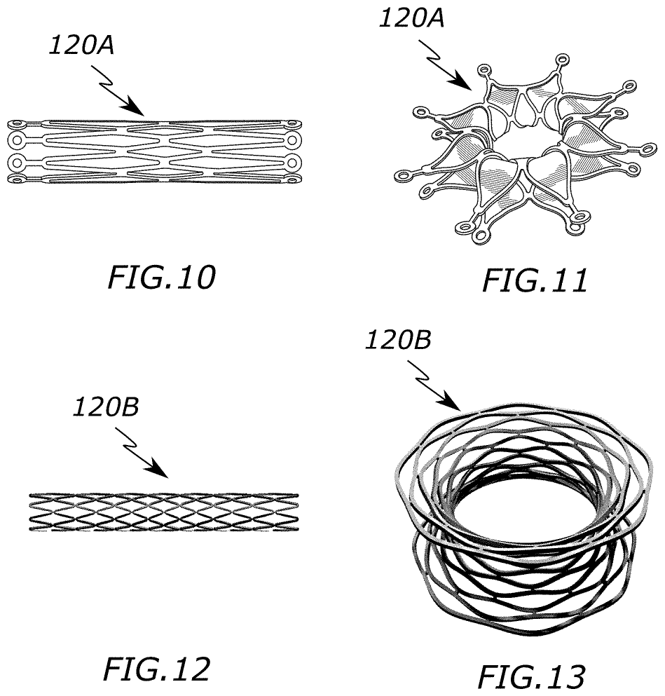

A variety of different shunt support structures 120 are possible. For example, FIGS. 10 and 11 illustrate one embodiment of a structure 120A in a radially compressed and radially expanded configuration, respectively. This structure 120A has loops or leaflets that self-bend or can be bent (e.g., via balloon inflation) generally perpendicularly to engage the tissue of each vessel. Another example shunt support structure 120B can be seen in FIGS. 12 and 13 in a radially compressed and radially expanded configuration, respectively. This structure 120B may expand in a manner similar to a rivet by decreasing in length and radially increasing in size at its proximal and distal ends. Further shunt support structures and details on structures 120A and 120B can be found in U.S. application Ser. Nos. 16/576,704 and 16/785,501, both of which are incorporated herein by reference. Additional shunt methods, techniques, and equipment can also be found in the aforementioned incorporated references.

The following embodiments and methods are discussed in the context of the previously described shunt creation technique and equipment. While only portions of the previously described equipment and procedures are discussed, it should be understood that any or all of the previously described equipment and procedures can be combined with those described below.

In the previous discussion of FIGS. 6-8, the puncturing guidewire 112 is advanced through the superior vena cava 12 and the right pulmonary artery 14, the crossing catheter 108 is advanced through the superior vena cava 12 and into the right pulmonary artery 14, and the delivery catheter 114 is advanced through the punctures of the vessels to deliver the shunt support structure 120. This procedure can be simplified by using a single catheter to both “steer” and dilate/cross the opening created by the puncturing guidewire 112. Such a device may be generally similar to the catheter shown in U.S. Pat. No. 10,076,638, herein incorporated by reference, but may further include a distal tip shape to dilate tissue openings (e.g., a tapered distal tip).

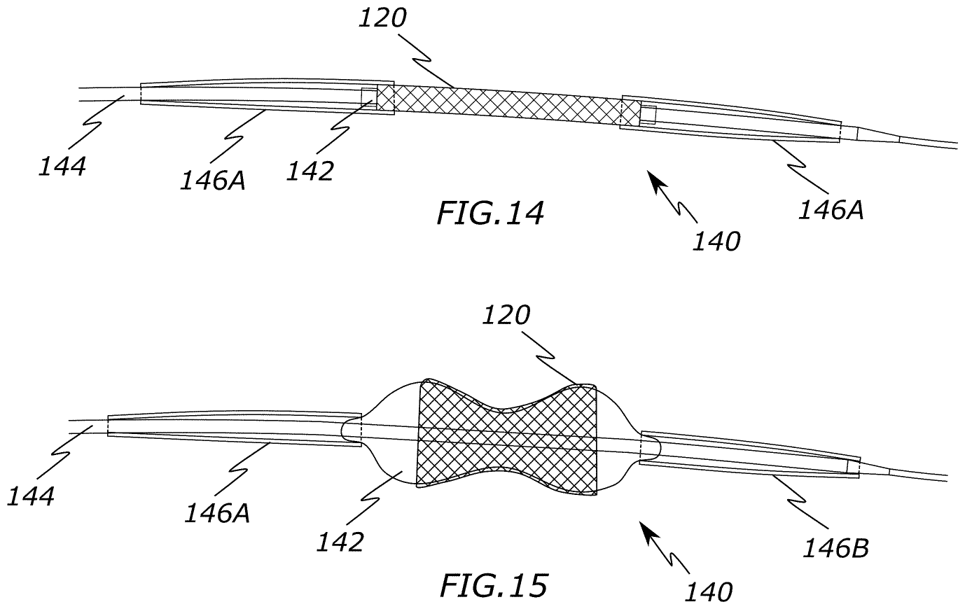

In the previous discussion of FIGS. 6-8, the puncturing guidewire 112 is advanced through the superior vena cava 12 and the right pulmonary artery 14, the crossing catheter 108 is advanced through the superior vena cava 12 and into the right pulmonary artery 14, and the delivery catheter 114 is advanced through the punctures of the vessels to deliver the shunt support structure 120. FIGS. 14 and 15 illustrate one embodiment of a delivery catheter 140 that can cross both vessels 12 and 14 without an overlying sheath disposed completely over the shunt support structure 120 during crossing.

Typically, delivery catheters for stent-like devices include an overlying sheath that completely covers the stent-like device until it is in position for being expanded, at which point the sheath is withdrawn. However, when a delivery device is positioned through the wall of two vessels (e.g., vessels 12 and 14), withdrawing the overlying sheath may pull one or more of the vessel walls, causing the vessels walls to reposition relative to the position of the underlying support structure 120. Hence, minimizing movement against the vessel's walls may help maintain a more consistent position of the vessel walls relative to the shunt support structure 120.

The delivery catheter 140 may include a proximal sleeve 146A and/or a distal sleeve 146B that are each positioned over only the proximal and/or distal ends of the shunt support structure 120 (e.g., 1-5 mm on each end) and radially compressed on a distal end of the catheter 140. A middle portion of the support structure remains uncovered by any protective barrier, such as a sleeve or sheath. This allows most of the shunt support structure 120 to pass through the openings of the vessels “bare”. The sleeves 146A and 146B may be conical in shape, decreasing in diameter away from the structure 120, and may be composed of a relatively soft polymer material.

In one example, the sleeves 146A and 146B are disposed over the elongated body 144 of the catheter 140 in a manner that allows them to slide away from the support structure 120 prior to or during expansion. The one or more of the sleeves 146A and 146B may freely move or slide over the elongated body 144, may be biased to positions covering the support structure 120 (e.g., via a spring or other compressible item positioned within the sleeves 146A and 146B or at either of their free ends), or may have a releasable locking mechanism that releases the sleeves 146A and 146B from a locked position to an unlocked and slidable position (e.g., via a pull wire).

In the example of FIGS. 14 and 15, a balloon 142 is included underneath the support structure 120. The proximal and distal ends of the balloon 142 can be shaped and positioned such that they push the sleeves 146A and 1466 away from the support structure 120 when inflated, so as to release the sleeves 146A and 146B from radially retaining the support structure 120. The balloon 142 may also include a tacky or adhesive layer on its outer surface to help further retain the support structure 120 in position on the delivery device 140 during positioning, but while also allowing the support structure 120 to be released during expansion.

Alternately, the proximal sleeve 146A may instead be an outer sheath or catheter with a similar distal position that extends back to a proximal end of the elongated body 144. This outer sheath functions similar to the proximal sleeve 146A except that it is longer. Hence, as the balloon 142 inflates, the outer sheath is proximally pushed back. A bias mechanism, such as a spring, may be connected between the proximal ends of both the outer sheath and the elongated body 144 so as to keep the outer sheath over at least a proximal end of the support structure 120. Additionally, the outer sheath allows the user to manually retract the outer sheath, if necessary, since it extends to the proximal end of the elongated body 144. The distal sleeve 146B may optionally be present in this embodiment.

Alternately, the sleeves 146A and 146B may be configured to remain in place without sliding, but instead at least partially tear as the balloon 142 expands. These sleeves 146A and 146B may be composed of a relatively thin material (e.g., urethane) and may include weakened areas or one or more cuts to promote tearing during expansion.

Alternately, the sleeves 146A and 146B may be configured to remaining in place without sliding or tearing but are instead configured such that the support structure 120 slides out of the sleeves 146A and 146B as the balloon 146A expands. The inner surface of the sleeves 146A and 146B may include a coating to reduce friction and allow slippage. The sleeves 146A and 146B may also be composed of a material that stretches as the balloon 142 expands, allowing the support structure to pull out of the sleeves 146A and 146B as the balloon expands 142.

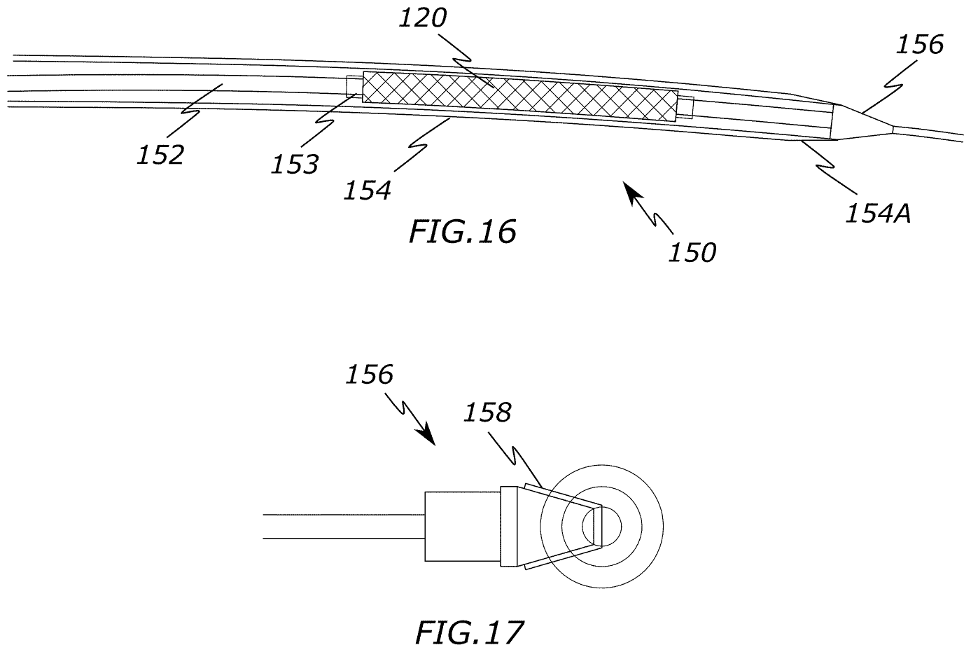

FIGS. 16 and 17 illustrate another embodiment of a delivery catheter 150 that can be used to both pierce the vessels walls of two vessels, such as the superior vena cava 12 and the right pulmonary artery 14, as well as deliver the support structure 120 to both vessels 12 and 14. Hence, instead of the need to use a separate puncturing guidewire or similar device and delivery catheter, only the delivery catheter 150 is needed for the puncture and support structure 120 delivery.

The delivery catheter 150 includes an elongated body 152 with a distal tip 156 configured for piercing vessel walls. In one example, the distal tip 156 includes one or more electrodes 158 that are connected to a power source to supply radiofrequency energy to create an opening in a vessel (e.g., the one or more electrodes 158 are electrically connected to an RF power supply via a proximal end of the catheter).

The delivery catheter 150 can also act as a dilator catheter by having a conical cone that decreases in diameter in the distal direction. Additionally, the delivery catheter 150 may have an outer sheath 154, and therefore to help with dilation, a distal portion 154A of the sheath 154 may be tapered, decreasing in thickness in a distal direction (e.g., along about 2-5 mm in length).

The delivery catheter 150 can also include a support structure 120 that is radially compressed over an inflatable balloon 153. An outer sheath 154 can be withdrawn proximally to expose the support structure 120 and the balloon 153 can be inflated.

In operation, the delivery device 150 is advanced with a vessel, such as the superior vena cava 12 such that its distal tip 156 is angled towards a target or snare catheter in an adjacent vessel, such as a right pulmonary artery 14. The one or more electrodes on the distal tip 156 are activate, e.g., applying radiofrequency energy, to thereby cause an opening in both vessels 12 and 14. The taper of the distal tip 156 and the taper of the distal portion 154A allow the catheter 150 to be pushed through both openings so that it is positioned in both vessels 12 and 14. Next, the outer sheath 154 is proximally withdrawn to expose the support structure 120. Finally, the balloon 153 under the support structure 120 is inflated to expand the support structure (or optionally the support structure is self-expanding). In this manner, the delivery catheter 120 may take the place of several other catheters with dedicated purposes.

As previously discussed in FIGS. 5-8, a puncturing guidewire 112, such as an RF guidewire, can be used to puncture or pass through both the superior vena cava 12 and the right pulmonary artery 14. One danger of using such a RF guidewire is the risk it will contact an unintended area of either of the two vessels 12 and 14 during a procedure, thereby damaging or even creating another opening in one of the vessels 12 and 14. Particularly, there is a danger of extending an RF guidewire longitudinally too far through one or more vessels, such that two openings are created in a vessel.

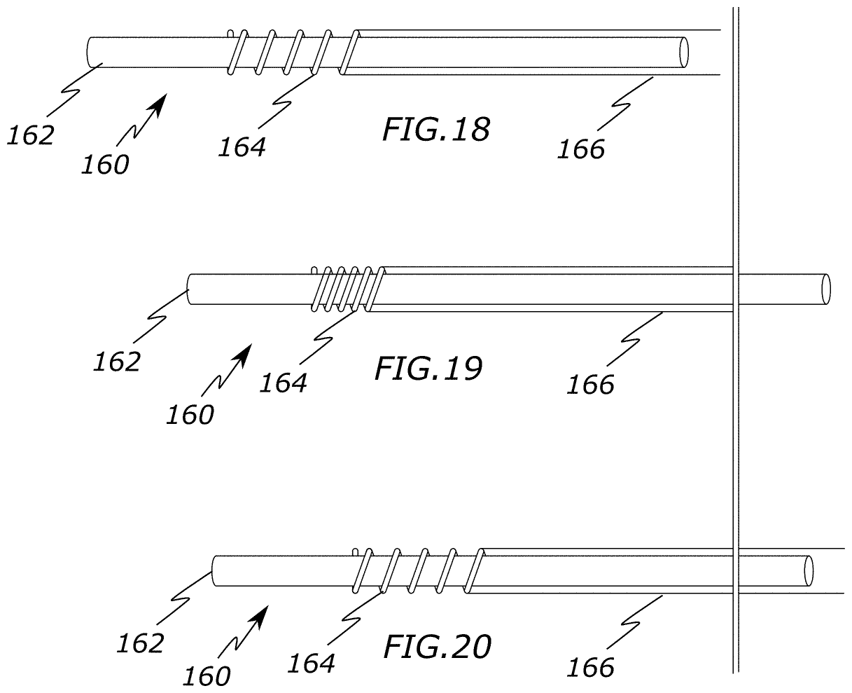

FIGS. 18-20 illustrates one embodiment of an RF puncturing guidewire 160 that includes a protective sheath 166 a distal end of an elongated RF wire body 162 to help protect from unintended lateral contact and unintended longitudinal contact. The sheath 166 is configured to maintain a position such that its distal end is either even with or extends beyond the distal end of the RF wire body 162, as seen in FIG. 18. The distal end of the RF wire body 162 includes one or more RF electrodes that are connected to a power supply, and the sheath 166 thereby prevents contact with tissue in that FIG. 18 position. Preferably, the sheath 166 has a tubular shape for maximum lateral protection, though other configurations are also possible, such as a braided tubular shape.

The sheath 166 is configured to be longitudinally slidable and biased to the FIG. 18 position. For example, a spring 164 or similar compressible element may be fixed to the RF wire body (e.g., at a proximal end of the spring 164) and to the sheath 166 (e.g., at a distal end of the sheath 166), causing the sheath 166 to bias distally. In this respect, the sheath 166 can be configured to longitudinally move only a predetermined distance (e.g., about 1 cm), which may prevent it from passing entirely through the second vessel (e.g., the right pulmonary artery 10).

As seen in FIG. 19, when the distal end of the RF puncturing guidewire 160 is pushed against tissue (e.g., a vessel wall), the sheath 166 moves proximally back only a predetermined distance as the RF wire body 162 pushes against and through a vessel wall (e.g., a stop). The predetermined distance can be configured so as to limit the travel of the RF puncturing guidewire 160, thereby preventing it from advancing it too far. As seen in FIG. 20, the sheath can also be pushed through the first vessel wall to cover the distal end of the RF wire body 162 until it is pressed against and through the adjacent vessel.

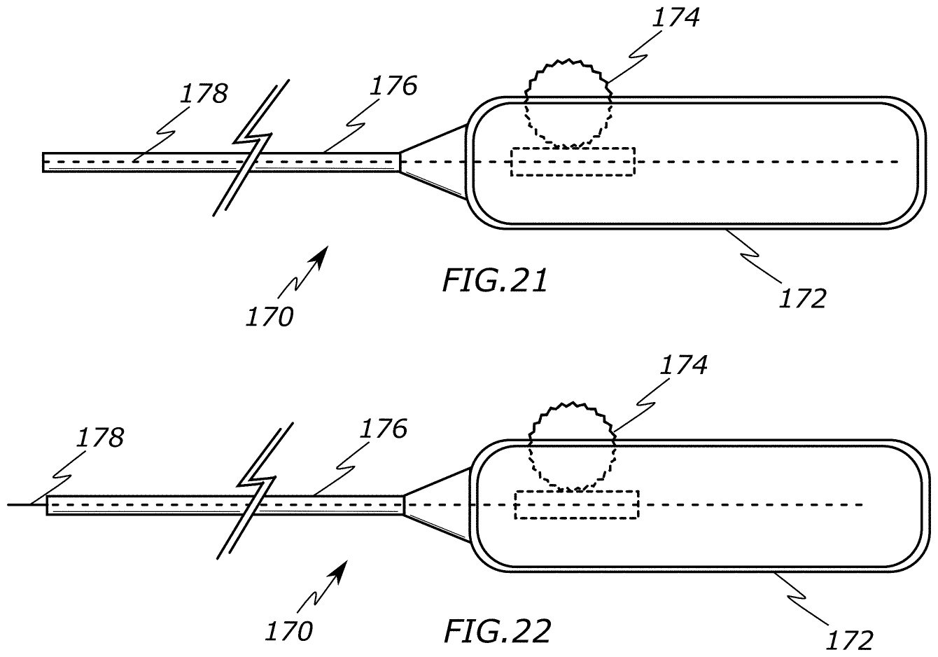

FIGS. 21 and 22 illustrate another embodiment of an RF guidewire assembly 170 that is configured to limit and/or control longitudinal movement of an RF puncturing guidewire to prevent it from distally extending completely through a second vessel (e.g., two walls of a right pulmonary artery 10). Specifically, a handle portion 172 includes a mechanism configured to move the RF puncturing guidewire 178 relative to its outer tubular sheath 176. In one example, the mechanism includes a thumbwheel 174 that engages a toothed track connected to the RF puncturing guidewire 178 such that rotation of the thumbwheel 174 moves the track and the guidewire 178 longitudinally. A limit mechanism or stop member can be positioned within the handle to prevent movement of the guidewire 178 beyond a predetermined distance that would otherwise puncture entirely through a vessel (e.g., 1 cm). Alternate movement mechanisms are possible, such as screw drive mechanisms or thumb sliders. The handle 172 and the guidewire 178 can be connected to an RF power source so as to allow the guidewire 178 to apply RF energy to the patient's tissue.

In practice, the user advances the tubular sheath 178 so that the distal end is in a desired target location. RF energy can be applied to the guidewire 178 so that its distal end can apply radiofrequency energy to tissue. The user can rotate the thumbwheel 174 to cause the RF puncturing guidewire 178 to contact the wall of a first vessel (e.g., superior vena cava 12), pass through its vessel wall, contact a second vessel (e.g., right pulmonary artery 10) and then pass through its wall.

In an alternate embodiment, the handle 172 can move the outer sheath 176 relative to the RF puncturing guidewire 178. This allows the user to advance the entire guidewire assembly 170 to be distally advanced until the distal end of the sheath 178 blocks further advancement.

Alternately or additionally, the guidewire assembly 170 may include a switch or circuit breaker mechanism that interrupts the RF current when the guidewire 178 is extended from the sheath 176 a predetermined distance (e.g., 1 cm). The switch or circuit breaker mechanism may be located within the handle 172 and can be actuated when a portion or feature on or connected to the guidewire 178 distally advances to the predetermined distance. In another embodiment, the switch may be an electrolytic segment of the circuit near or in electrical communication with one of the electrical contacts of the puncturing guidewire 112 or snare, such that as the electrical contacts of the puncturing guidewire 112 contact the snare catheter (e.g., the shield or loops), the electrolytic segment or fuse dissolves, breaking the circuit.

In another embodiment, any of the piercing guidewires discussed in the specification may be connected to an RF energy source with a timer configured to activate for only a length of time sufficient to pierce through one wall of the first vessel (e.g., superior vena cava 12) and/or one wall of the second vessel (e.g., right pulmonary artery 14). For example, the RF energy may be activated for only 0.5 second, 1 second, 1.5 seconds, or two seconds. In this manner, the RF energy can be quickly turned off to prevent unwanted damage (e.g., puncturing entirely through opposite walls of a vessel).

As previously discussed with regard to FIGS. 6-8, a target or snare catheter 104 can be used to capture a puncturing guidewire 112. One challenge with using a snare catheter in this manner is that it may be difficult to maintain the position of its loops 104A so that the puncturing guidewire 112 can be threaded through. Additionally, once through the loops 104A, the puncturing guidewire 112 may be accidentally advanced through the opposite side of the vessel it entered (i.e., entirely through the vessel). The following embodiments address one or more of these challenges.

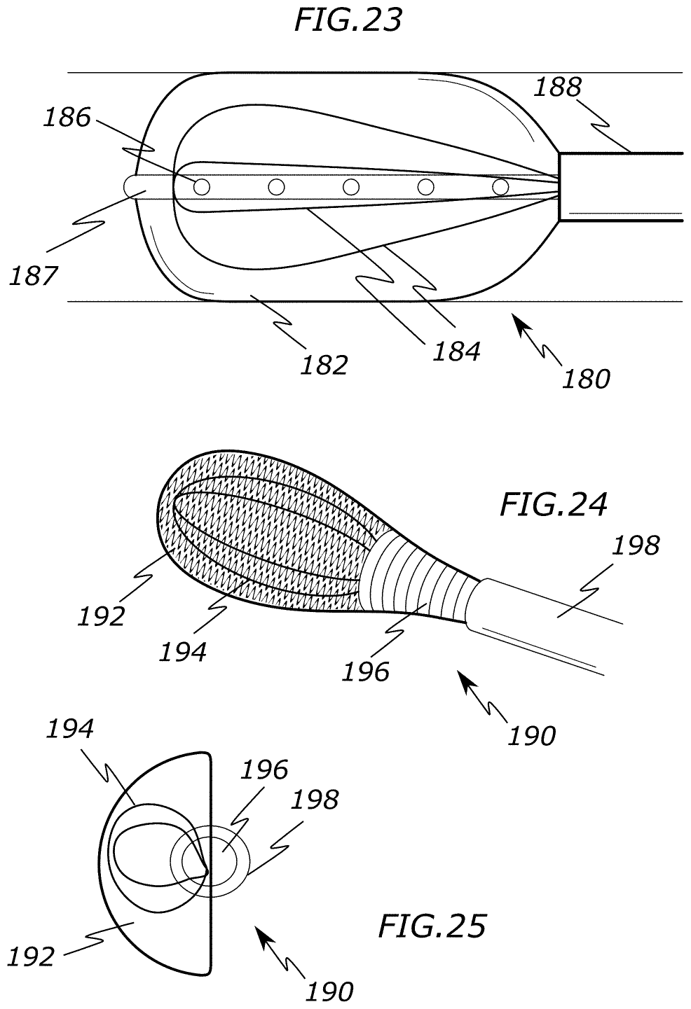

The snare catheter 180 shown in FIG. 23 includes an inflatable balloon 182 that can be inflated to engage the walls of the vessel (e.g., right pulmonary artery 14) so that its distal end can be locked into place within the vessel. The balloon 182 can be located at the distal end of an elongated catheter body 187, which further includes one or more apertures 186 in communication with a fluid passage through the body 188 that allows inflation of the balloon 182. The catheter body 187 can be moved into and out of an elongated tubular sheath 188.

One or more snare loops 184 (e.g., two loops) are positioned at the distal end of the elongated catheter body 187. This can be achieved in several ways. For example, the loops 184 can be fixed to the elongated catheter body 187 and positioned within the balloon 182 such that the balloon 182 inflates around the loops. In another example, the loops 184 may be fixed to the elongated catheter body 187 and positioned outside of the balloon 182 such that the loops remain on an outer surface of the balloon 182 when inflated. In another example, the loops may be positioned outside of and adjacent to the balloon 182 but are connected to a separate elongated body or pusher that allows the loops 184 to move independently of the balloon 182. In another example, the loops 184 can be embedded, adhered to, or bonded to the material of the balloon 182.

In practice, the distal ends of the sheath 188 and catheter body 187 can be positioned at a desired location in a vessel (e.g., right pulmonary artery 14), the balloon 182 can be inflated to engage the walls of the vessel, a puncturing guidewire 112 can be advanced through the loops 184 (and optionally through the balloon 182, and the loops 184 can be at least partially retracted into the sheath 188 to grab the puncturing guidewire 112.

Optionally, the balloon 182 may be composed of a puncture resistant material that resists puncture from the puncturing guidewire 112. For example, only one side may be composed of a puncture resistance material when the loops 184 are located within the balloon 184, allowing the puncturing guidewire 112 to pass through one side of the balloon 184 but not its opposite side. In embodiments with the loops 184 being located outside the balloon 182, the entire balloon may be composed of puncture resistant material. The puncture resistant material may be a hardened polymer or flexible material containing one or more metal strands or panels.

FIGS. 24 and 25 illustrate an alternate embodiment of a snare catheter 190 that includes a rear shield 192 that extends behind a plurality of wire snare loops 194 and blocks a puncturing guidewire 112 from passing entirely through the vessel it is deployed in (e.g., right pulmonary artery 14). Both the snare loops 194 and the shield 192 may be fixed to the end of an inner elongated catheter body 196 which can be extended out of and pulled into an outer tubular sheath 198.

The shield 192 may be composed of a plurality of woven or braided wires, textile, a polymer sheet (e.g., polyurethane), silicone, or similar materials. The shield 192 may also be composed of a shape memory frame (e.g., a Nitinol wire) that allows the shield 192 to expand to its desired shape. The shield 192 may also expand from a radially compressed configuration to an expanded configuration having a variety of different shapes. For example, the shield 192 may expand to an oval, planar shape. In another example, the shield 192 may expand to a curved shape across the axis of the device to conform to the curvature of the vessel it is deployed in, as seen in the end view of FIG. 25.

In one embodiment, the shield 192 can be configured to turn off radiofrequency energy being supplied to a puncturing guidewire 112 that uses RF energy. For example, the shield 192 may be composed of an outer electrically insulated layer and an inner conductive layer so that when the puncturing guidewire 112 punctures through, it creates electrical contact with the conductive layer. The conductive layer and therefore the snare catheter 190 may be connected to an RF power supply that is configured to interrupt the RF power to the puncturing guidewire 112.

The inner catheter 196 may also include a funnel/cone portion at the distal end of its body and proximal of the shield 192 and loops 194 to help radially compress these structures as the inner catheter 196 is pulled proximally back into the outer sheath 198. For example, the funnel may be composed of one or more coiled wires, a braided mesh cone, or a polymer cone.

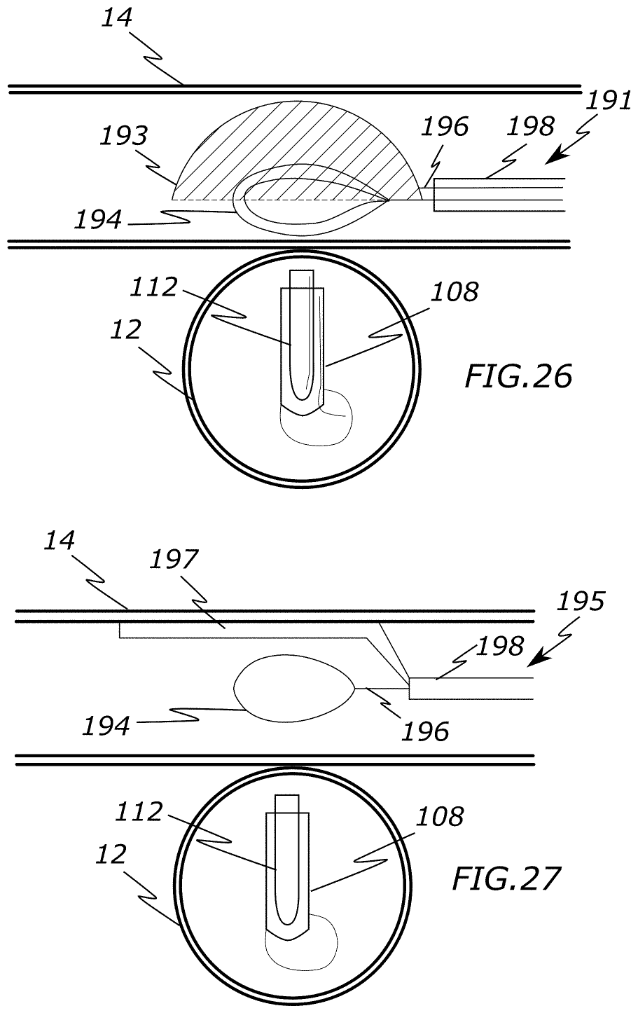

FIG. 26 illustrates an embodiment of a snare catheter 191 that is generally similar to the previously described snare catheter 190 but has a shield 193 forming a circular diameter with a concave interior as opposed to the more oval shape of shield 192. In other words, the shield 193 is hemispherical with an interior space positioned at least partially around loops 194.

FIG. 27 illustrates an embodiment of a snare catheter 195 that is generally similar to the previously described snare catheter 190 but has a generally planar shield 197. The shield 197 can have a variety of different planar shapes, such as a square, rectangle, circle, or oval shape. Optionally, the “plane” of the shield 197 may also have a slight curve and the axial direction of the catheter 195, thereby forming a partial tubular shape.

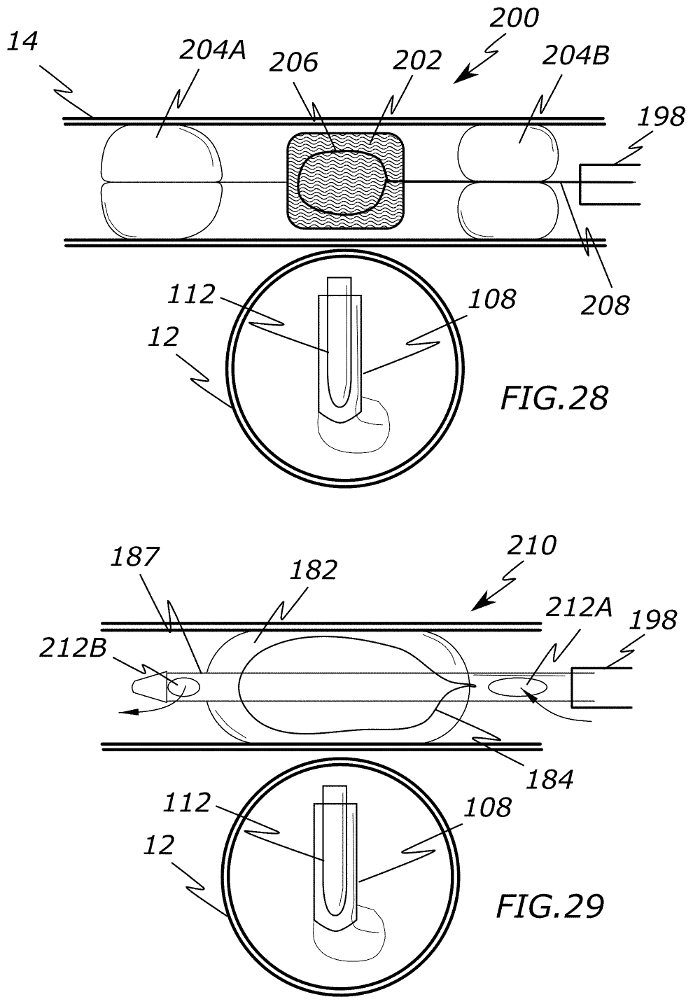

FIG. 28 illustrates an embodiment of a snare catheter 200 that is generally similar to the previously described snare catheter 190 but includes an anchoring mechanism to anchor the shield 202 and snare loop 206 in a desired position within a vessel (e.g., right pulmonary artery 14).

In one example, an elongated inner catheter 208 includes one or more distal balloons 204A and one or more proximal balloons 204B that are spaced on either side of the shield 202 and snare loop 206. The inner catheter 208 includes one or more inflation lumens that are configured to connect to a fluid supply, thereby allowing the balloons to be inflated. Each of the balloons 204A can be a single balloon that entirely expands with in the vessel 14 or can each include a plurality of balloons (e.g., two, three, four, or five balloons). By using a plurality of balloons, it may be possible to include spaces or perfusion passages across the balloons to allow for blood flow during inflation.

As in any of the previous embodiments, the snare loop 206 can be fixed to the shield 202 or the snare loops 206 can be connected to a separate elongated wire or body that allows it to move independently of the shield 202.

In practice, the distal end of the inner catheter 208 is positioned at a desired shunt creation location, outside of the outer sheath 198. Next, the one or more distal balloons 204A and one or more proximal balloons 204B are inflated to engage the walls of the vessel (e.g., right pulmonary artery 14), distally and proximally of the expanded shield 202 and snare loop 206. The puncturing guidewire 112 is then advanced through another vessel (e.g., superior vena cava 12), into the prior vessel (e.g., right pulmonary artery 14), through the snare loop 206, and is prevented from further advancement by the shield 202. Finally, balloons 204A and 204B are deflated and the inner catheter 208 (or the wire connected to the snare loop 206) is at least partially retracted into the outer sheath 198 to grasp the puncturing guidewire 112.

Any of the embodiments relating to a target or snare catheter may include perfusion features or passages to allow blood to flow around any blockages that are created. While these perfusion features may be particularly desirable for embodiments with balloons (e.g., snare catheter 180 in FIG. 23 or snare catheter 200 in FIG. 28), it may also be desirable in embodiments with a shield as well, since these shields may at least partially block blood flow through the vessel.

As previously discussed for the snare catheter 200, one way to achieve perfusion passages is to provide two or more balloons at a particular location that, when inflated, create gaps or longitudinal passages between themselves. Another technique can be seen in the snare catheter 210 in FIG. 29 which includes a proximal perfusion opening and a distal perfusion opening 212B that both connect to a perfusion passage or channel therebetween in the inner catheter 187. This snare catheter 210 is generally similar to the snare catheter 180 in FIG. 23, but the perfusion channel and openings 212A and 212B can be used on any of the snare catheter embodiments described herein, including those with balloons that also have perfusion passages between themselves.

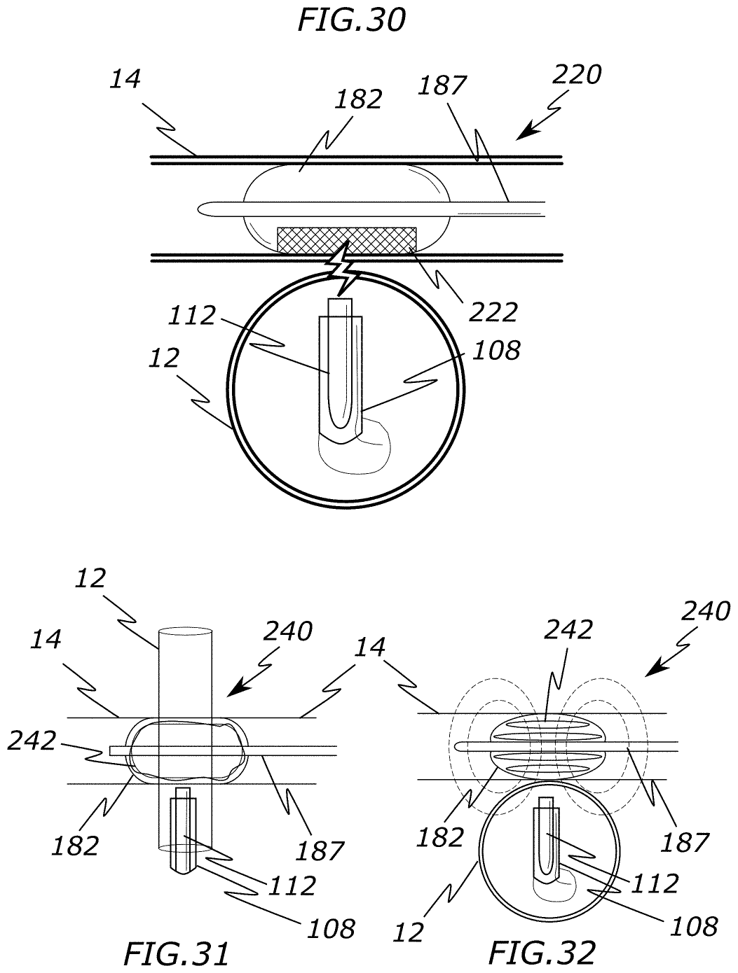

As previously discussed, it can be undesirable for radiofrequency energy from a puncturing guidewire 112 to damage unwanted areas of the patient. FIG. 30 illustrates one embodiment of a snare catheter 220 that helps maintain the RF energy between only the puncturing guidewire 112 and the snare catheter 220 by including one or more RF electrodes 222 in the snare catheter 220. For example, the electrode 222 may be embedded within a balloon 182, a shield, or any component of the snare catheter embodiments of this specification. The one or more electrodes 222 may have an opposite polarity to the electrodes on the distal end of the puncturing guidewire 112 and may also be connected to the RF energy source outside of the patient. Hence, the RF energy takes the path of least resistance to the electrode 222, thereby avoiding other tissue that is not intended to be damaged. The electrodes can be strips of conductive material on or embedded in the balloon 182 (or other component) or can be a plurality of wires arranged in a pattern (e.g., braided).

The snare catheter embodiments of this specification may also include mechanisms for sensing the position of the snare catheter and/or aligning puncturing guidewire 112 with the snare catheter. FIGS. 31 and 32 (side and top views, respectively) illustrate one example of such a snare catheter 240 that creates a magnetic field that can be used for either positioning or self-aligning purposes. In this example, the snare catheter 240 is generally similar to the snare catheter 180 in FIG. 23 except that one or more coils of conductive wire 242 is located within, on, or embedded into the balloon 182. The one or more coils of conductive wire 242 are connected to a power source via the proximal end of the catheter 240, allowing current to selectively pass through the one or more coils 242 and generate a magnetic field.

The magnetic field can be used in two possible ways. First, the puncturing guidewire 112 may include one or more magnetic sensors that can sense the magnetic field, allowing the puncturing guidewire 112 to be better aligned with the snare catheter 240. For example, the one or more sensors may sense the magnitude of the magnetic field on each side of the puncturing guidewire 112 and/or may sense the polarity of the magnetic field, thereby providing additional data to achieve a desired orientation. Second, the puncturing guidewire 112 may have its own magnets or ferrous material that is attracted to the magnetic field generated by the one or more coils of conductive wire 242. This may provide physical force and guidance to better align the puncturing guidewire 112 with the snare catheter 240. Either of these two sensing/aligning features or both of these features can be used.

The coil 242 may also be incorporated into other structures, such as a shield or catheter body. Alternately, either a balloon or shield may include one or more permanent magnets to provide similar functionality. Alternately, ferrous material can be incorporated into the balloon or shield and the puncturing guidewire 112 may include permanent magnets or an electromagnet (e.g., conductive wire coil).

As previously discussed, one challenge of a shunt procedure between vessels, particularly between the superior vena cava 12 and right pulmonary artery 14, is directing the puncturing guidewire 112 through the vessel walls at the desired location and at the desired angle. Further, as the puncturing guidewire 112 is advanced out of the outer steerable catheter 110 (or out of the crossing catheter 108 within the steerable catheter), it may cause the steerable catheter 110 to deflect from the intended position and angle.

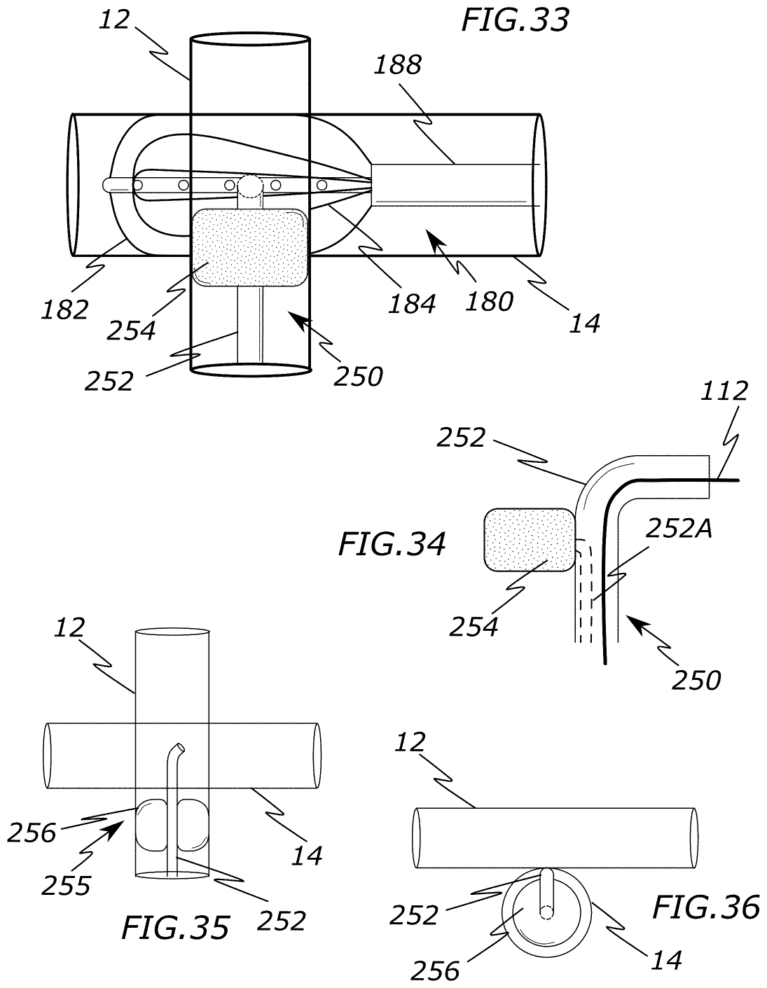

One approach to maintaining the position of the steerable catheter 110 during a procedure is to include an expandable member on a side of the catheter opposite of which it bends forward so as to brace the distal end of the catheter 110 in place. For example, FIGS. 33 and 34 illustrate a steerable catheter 250 having an elongated tubular body with an inflatable balloon 254 that is positioned on an outer surface of the catheter body 252, opposite of the distal opening of the catheter body 252. When the steerable catheter 250 is bent in a first direction, the balloon 254 can be inflated via inflation passage 252A, either prior to or after the bending. The balloon 254 expands in a direction opposite of the bend and braces the back side of the catheter body 252 which allows the puncturing guidewire 112 to be advanced out in a predictable direction and location. The steerable catheter 250 generally comprises an elongated tubular body that includes mechanisms to allow the distal end of the catheter to bend via user controls on a proximal end of the catheter.

FIGS. 35 and 36 illustrate a similar steerable catheter 255 in which one or more balloons 256 inflate on multiple sides of the catheter body 252 to center the catheter body 252 within the vessel 12. Again, this helps provide an anchored position for the steerable catheter 255 that allows for a more predictable location and direction advancement of the puncturing guidewire 112. The one or more balloons 256 can be a single balloon that extends entirely or nearly entirely around the circumference of the catheter body 252, or can be two or more balloons (e.g., 3, 4, or 5 balloons).

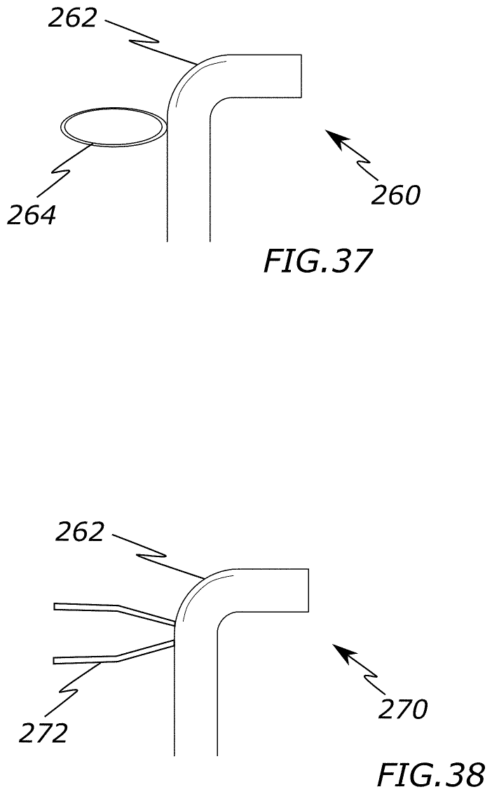

FIG. 37 illustrates another embodiment of a steerable catheter 260 that includes an expandable wire frame or structure 264 that can expand perpendicularly to an axis of the catheter body 262 from a side opposite the bent opening of the body 262. In one example, the expandable wire structure 264 is composed of a shape memory material (e.g., Nitinol) and is shape set to expand to the desired perpendicular position. The wire structure 262 can be a ring shape (e.g., circular, square, etc.). Alternately, the wire structure can be one, two, three, four, or more arms 272, as seen in the steerable catheter 270 in FIG. 38. Each arm can be composed of a shape memory material (e.g., Nitinol) that is biased outwards in a direction generally perpendicular to the body 262. Each arm 272 can be a single wire (e.g., generally straight or bent) or each arm 272 can be a loop of wire (e.g., circular, oval, square, rectangular, etc.).

While the embodiments of the previously discussed FIGS. 33 through 37 are contemplated for use on a steerable catheter through which the puncturing guidewire can be advanced through, other components may also use these features. For example, the snare catheter 104 may also include one or more of these centering or positioning features.

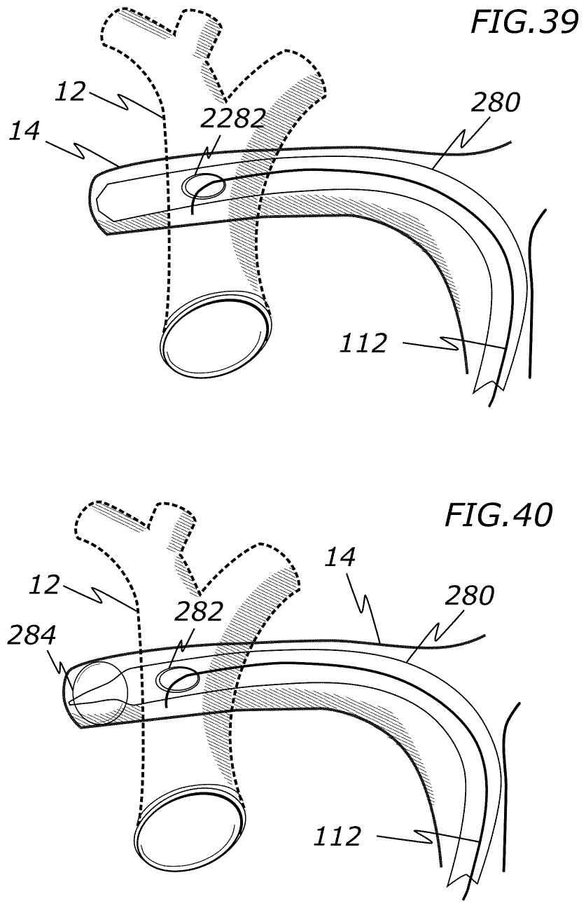

Turning to FIG. 39, a catheter 280 or elongated catheter body having a side aperture 282 can also be used with the shunt creation methods of this specification. The catheter 280 includes at least one lumen within it that is in communication with the aperture 282. The aperture 282 may be located in the sidewall of the catheter, just proximal of the distal end of the catheter 280. For example, the aperture may be about 1-2 cm from the distal end of the catheter 280. The aperture 282 may also have a general diameter of about 0.1-0.5 cm.

In one embodiment, the catheter 280 is configured to form a curve through its distal end to conform to the right pulmonary artery 14 and help brace it during a procedure. In one example, about 5 to 15 cm of the distal end has a curve of about 60-90 degrees relative to the remaining proximal portion of the catheter 280.

In one example use, seen in FIG. 39, the catheter 280 can be advanced into the right pulmonary artery 14 so that the aperture 282 aligns with the superior vena cava 12. Next, a puncturing guidewire 112 is advanced through the lumen of the catheter 280, out the aperture 282, and into the superior vena cava 12.

Optionally, the catheter 280 may include an anchoring device to help brace or maintain its position within the right pulmonary artery 14. One such anchoring device is a balloon 284 that is positioned at the distal end or tip of the catheter 280, as seen in FIG. 40. This balloon 284 is configured to be inflated to a size that engages the vessels walls (e.g., via an inflation lumen in the catheter 280). Alternately or additionally, the catheter 280 may include a balloon, ring, expandable braided mesh, or arms extending from the outer surface of the catheter wall, directly behind the aperture 282.

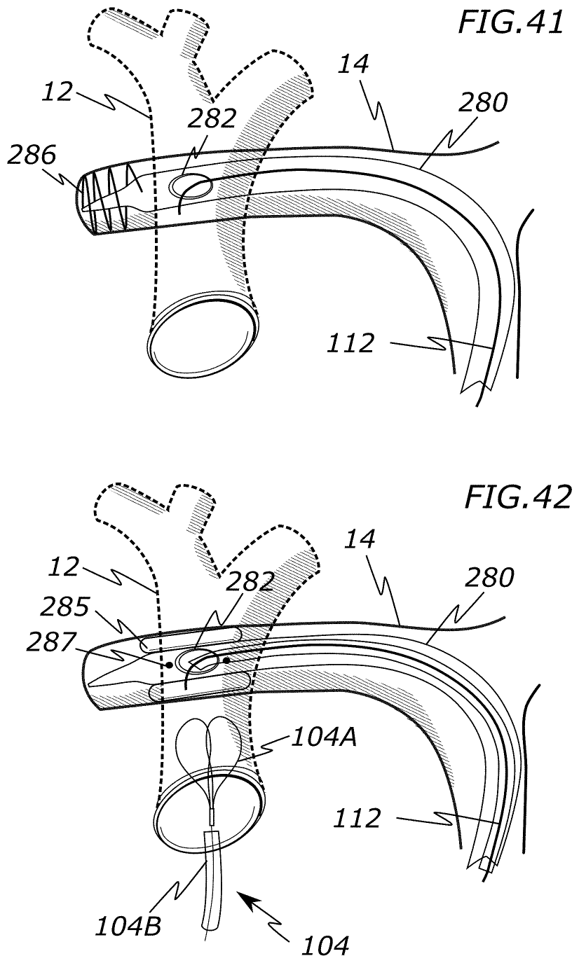

FIG. 41 illustrates another anchoring device comprising wire framework comprising a wire 286 that is attached to and radially expands from a distal end of the catheter 280 to engage the walls of the vessel. The wire may be composed of shape memory material (e.g., Nitinol) and shape set to a desired shape. The shape may include a helical coil, as seen in the figure, a plurality of loops, a plurality of arms, or similar shapes.

FIG. 42 illustrates another anchoring device comprising one or more centering balloons 285 positioned near or adjacent to the aperture 282 so as to position the catheter 280 near a center of the right pulmonary artery 14. Hence, the one or more centering balloons may help both anchor and position the catheter 280 to a position that allows access to the superior vena cava 12. However, the one or more centering balloons 285 may include any of the other anchoring devices previously discussed, as well.

In one example, the one or more balloons 285 is a single “C” shaped balloon that is positioned around the circumference of the catheter 280 at the location of the aperture 285 but leaving the aperture 285 uncovered. In another example, a plurality of cylindrical balloons can be used in a similar position to achieve the “C” shape.

Additionally, radiopaque markers 287 may be included adjacent the aperture 282 in this embodiment or any of the other embodiments. For example, a first marker 287 can be located just distal of the aperture 282 and a second marker 287 can be located just proximal of the aperture 282. Alternately or additionally, markers 287 can be located above or below (i.e., on the same circumference of the catheter 280) of the aperture 282.

As also seen in FIG. 42, a snare 104 (or any of the other snare embodiments of this specification, including those with shields or other safety measures that prevent completely passing through a vessel, such as the embodiment shown in FIG. 26) can be used in the superior vena cava 12 to snare or capture the puncturing guidewire 112. This snare 104 can be used in this manner with any of the previous examples/embodiments.

Again, while the catheter 280 in FIG. 42 is shown in the right pulmonary artery 14, this catheter may also be used in the superior vena cava 12 instead, as any of the embodiments of this specification can be reversed in this manner. In such an arrangement, any of the target/snare catheters described in this specification may be used.

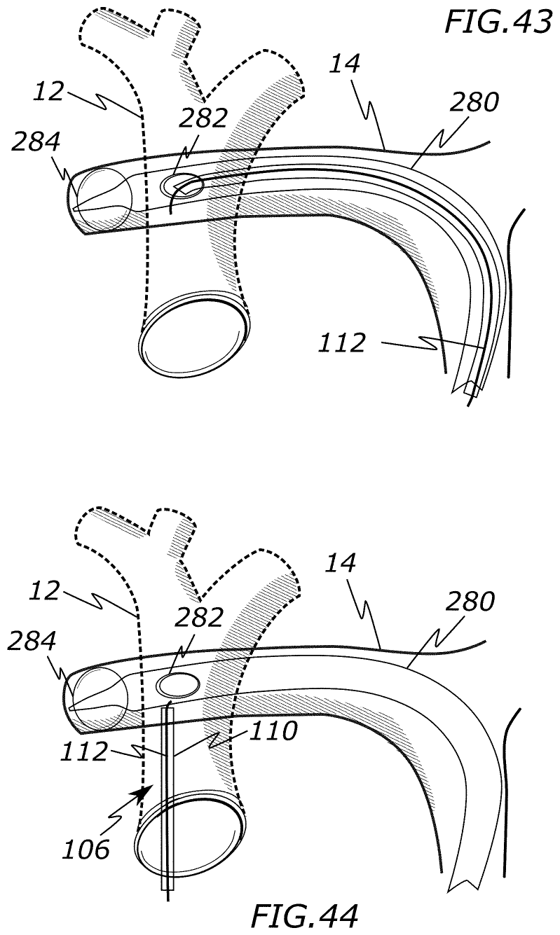

It may be helpful to provide an additional mechanism to help direct the puncturing guidewire 112 out of the aperture 282 in a desired direction. For example, the lumen of the catheter 180 may include a curved or ramped surface near the aperture 282 that is configured to help direct the distal end of the guidewire 112 out of the aperture 282. In another example, the puncturing guidewire 112 may include a balloon, wire loop, or wire arms, extending from one side of its body. In another example, a steerable catheter 110 may be advanced through the lumen of the catheter 280, along with the puncturing guidewire 112, as seen in FIG. 43. In this respect, the distal end of the steerable catheter 110 can be turned or directed so that its distal opening faces or extends out of the aperture 282.

Alternately, the catheter 280 may be used as a target catheter, similar to the previously discussed snare catheter, such that the puncturing guidewire 112 is advanced from the superior vena cava 12 into the right pulmonary artery 14, as seen in FIG. 44.

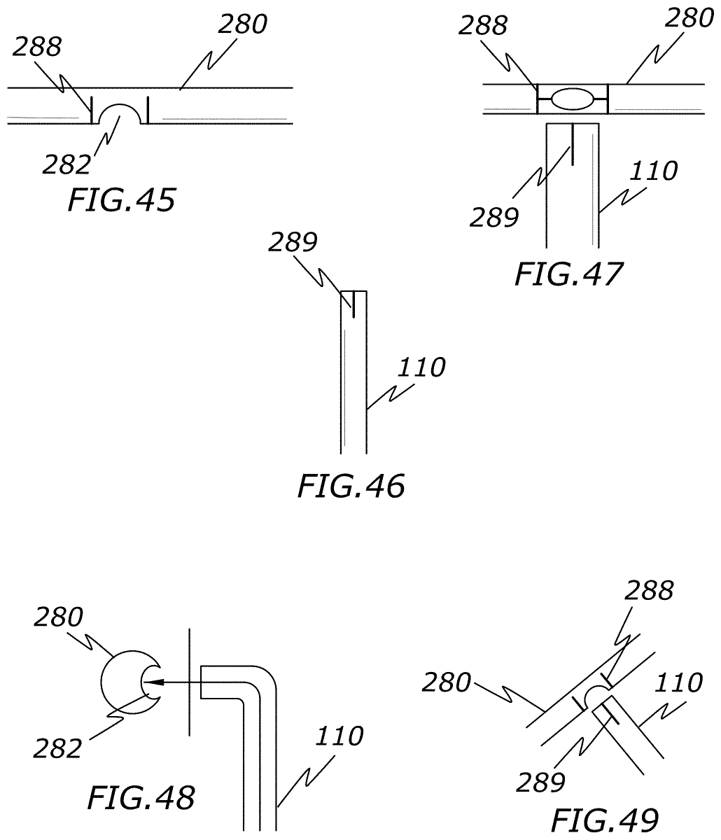

In such an arrangement, it may be desirable to include radiopaque markers on the catheter 280 and on the steerable catheter 110 (or alternately a crossing catheter 108). In one example seen best in FIGS. 45-49, the catheter 280 includes one or more radiopaque marker 288 that are located proximally adjacent and distally adjacent of the aperture 282. For example, the markers 288 may include a first and second line extending perpendicular to the axis of the catheter 280. Additionally or alternately, the markers 288 may include lines parallel to the axis of the catheter 280. The steerable catheter 110 may also include one or more radiopaque markers 289 that allow the user to help line up the distal end of the catheter 110 with the apertures 288 of catheter 280. In one example, the marker 189 is one or more (e.g., 2 or 4) radiopaque lines that are aligned with the axis of the steerable catheter 110. In the case of 2 markers 289, they can be located at about 180 degrees from each other and immediately adjacent to the distal end of the catheter 110. In the case of 4 markers 289, they can be located at about 90 degrees from each other and immediately adjacent to the distal end of the catheter 110.

In practice, the user can view both markers 288 and 289 and then align the markers 189 of the steerable catheter 110 with those markers 288 of the catheter 280. Once aligned (e.g., FIGS. 47-49), the puncturing guidewire 112 can be advanced out of the steerable catheter 110 and into the aperture 282 of catheter 280.

In another embodiment, the catheter 280 may include echogenic markers in similar positions as any of the previously discussed radiopaque markers, either instead of or in addition to the radiopaque markers. The echogenic markers allow a physician to utilize intracardiac echo imaging to monitor and then adjust the position of either of the catheters involved in the procedure.

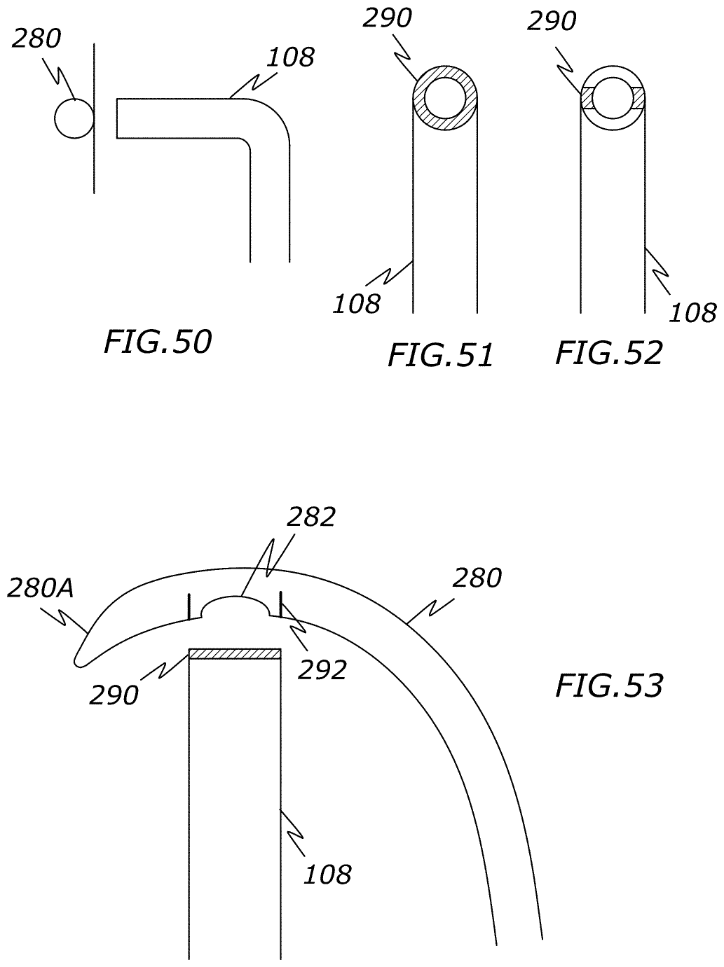

As previously discussed, the catheter 280 can be connected to with a steerable catheter 110 or flexible crossing catheter 108 (or a catheter with both abilities), via a puncturing guidewire 112 passing from either the superior vena cava 14 or right pulmonary artery 14. In either method, a magnetic connection mechanism can be used to help connect to the aperture 282, as seen in FIGS. 50-53. For example, the crossing catheter 108 may include a magnetic ring 290 located at or near the distal edge of the catheter 108. The ring 290 may have magnetic material extending entirely around the distal opening of the catheter 108 as seen in FIG. 51 or the ring 290 may have several discrete areas of magnetic material at locations around the distal opening of the catheter 108, as seen in FIG. 52 (e.g., at least two locations 280 degrees apart from each other).

The catheter 280 may include magnetic material 292 (or ferrous material) near or around the aperture 282. For example, the magnetic material 292 may be two lines or areas proximally and distally adjacent to the aperture 282. Preferably, the magnetic material 292 is spaced apart a similar distance as that of magnetic material 290 on the crossing catheter 290 and configured to attract each other (e.g., opposite polarities), allowing the two areas of magnetic material 290, 292 to align and engage with each other as the tip of the catheter 108 is advanced toward the aperture 282.

The catheter 280 may also include an elongated tip 280A to help position and brace the catheter 280 in a desired position to achieve a magnetic connection.

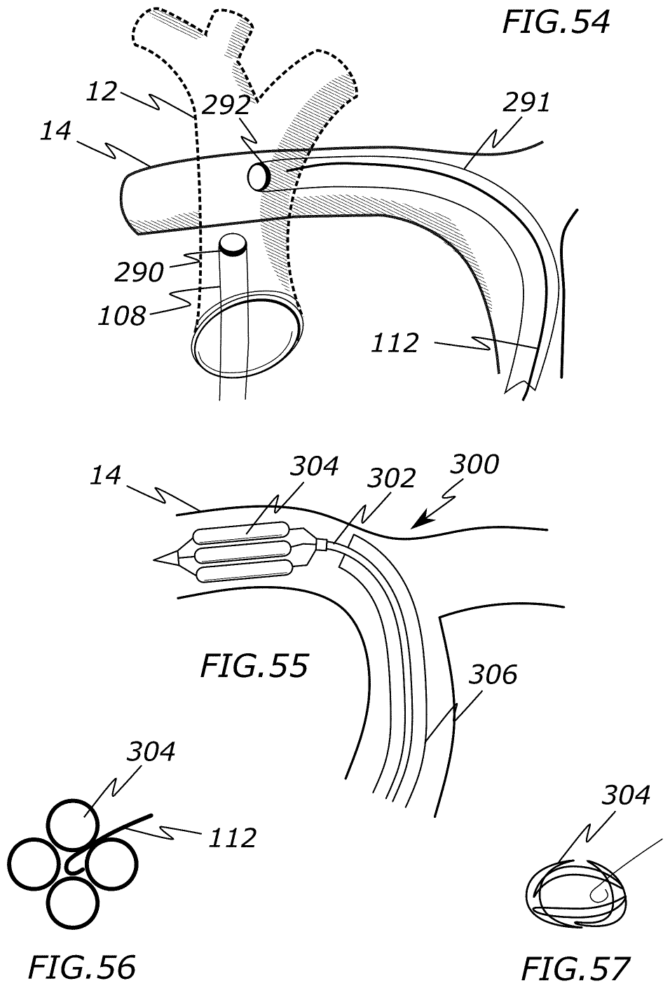

The magnetic material 290, 292 and previous configuration may be included on a variety of different catheter configurations, especially those described in the present specification. For example, two catheters 291, 108 with openings directly on their distal ends can be configured with the magnetic material 290, 192, as seen in FIG. 54. One of more of the catheters 291 and 108 may be steerable (as well as configured for crossing). Hence, the puncturing guidewire 112 can be advanced through either of the catheters 291, 108 and one of the catheters that is configured for crossing/dilating (e.g., crossing catheter 108) can move through the puncture, causing the magnetic material 290 to align with magnetic material 292, connecting the lumens of the two catheters.

FIGS. 55-57 illustrate another embodiment of a target or snare catheter system 300 that captures a distal end of a puncturing guidewire 112 via a plurality of balloons 304. When the puncturing guidewire 112 is positioned between the balloons 304 and the balloons 304 are deflated, they at least partially engage or wrap around the end of the guidewire 112, allowing the elongated catheter body 302 and balloons 304 to be withdrawn into the outer sheath 306, thereby capturing the guidewire 112.

The balloons 304 are positioned at the distal end of an elongated catheter body 302 which includes one or more lumens configured to inflate the balloons 304. The balloons 304 can have a variety of different shapes, including longitudinal cylindrical shapes, as seen in the figures. Preferably, the balloons 204 are positioned adjacent to each other so that after inflation they contact one another but also allows for some space between them so that the guidewire 112 can pass between them and into the space. In one example, the balloons 304 may be supported on a framework (e.g., of tubes or wires) with no central catheter member within the balloon group or alternately, a very small diameter tube/body that allows spacing between it and the balloons 304. The catheter system 300 includes at least two balloons, but three, four, five, six or more balloons 304 are also possible.

FIG. 56 illustrates the guidewire 112 moving into the central space between four inflated balloons after puncturing the walls of the vessels. Once positioned, the balloons 304 are deflated, as seen in FIG. 57, which cause the balloon material to partially wrap around the guidewire 112. The elongated catheter body 302 and balloons 304, along with the captured guidewire 112 are retracted into the outer sheath 306 to further lock the position of the guidewire 112.

This specification primarily discusses embodiments of the present invention with regard to a shunt connecting a right pulmonary artery to a superior vena cava. However, shunts can be created at other locations for similar purposes.

In one example, a main pulmonary artery (PA) is shunted to the right atrium or atrial appendage (RAA). In this method, a right-to-right shunt from a region of higher pressure in the PA is connected to a region of lower pressure in the RAA. Doing so utilizes the high compliance of the RAA to “absorb” additional volume received from the shunt since the RAA is a naturally compliant reservoir. An additional benefit may arise from the fact that the RAA and the main PA are both inside the pericardium and, therefore, would contain any leaks resulting as a complication of an improperly seated shunt. Another benefit may be that the risk of puncturing the aorta is minimized.

In another example, a connection made between a pulmonary artery (PA) and a pulmonary vein (PV) may be used to treat pulmonary hypertension or right heart failure/dysfunction. To reduce the total pulmonary vascular resistance and the afterload of the right ventricle, a shunt is created between a right pulmonary artery (RPA) and a right pulmonary vein (RPV). Alternatively, the shunt could be placed between a left pulmonary artery (LPA) and a left pulmonary vein (LPV).

In another example, a connection is created between a pulmonary artery (PA) and a left atrial appendage (LAA), in order to treat pulmonary hypertension, right heart failure/dysfunction, or atrial fibrillation, which reduces the total pulmonary vascular resistance and the afterload of the right ventricle. An added benefit to the reduced right ventricular afterload is the washout of the LAA in those patients that are at risk of stroke.

In yet another example, a shunt is created between a pulmonary vein (PV) and superior vena cava (SVC) to treat heart failure. This may particularly help treat elevated left atrial pressures causing fluid to back up in the lungs.

In yet another example, a plurality of shunts at different locations, such as any of the previously discussed locations can be used. For instance, there may be a benefit to placing an RPA-SVC shunt as well as an atrial shunt in certain populations. The RPA-SVC shunt would help reduce RV afterload and the LA shunt would help reduce PVR while keeping LA pressure and LV filling pressure low. To the same effect, there may be a benefit to the combination of the RPA-VC, intra-atrial, and arteriovenous peripheral shunt in certain patients.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1-16. (canceled)

17. A snare catheter, comprising:
an elongated catheter body;
one or more snare loops connected to a distal end of the elongated catheter body;
a shield connected to a distal end of the elongated catheter body; the shield being positioned on one side of the one or more snare loops and configured to resist being pierced by a puncturing guidewire.

18-22. (canceled)

23. An RF catheter system comprising:
a puncturing guidewire configured to puncture tissue with RF energy;
a snare catheter having an elongated body;
one or more RF electrodes connected at a distal end of the elongated body; and,
an RF power source connected to the one or more RF electrodes and the puncturing guidewire.

24-36. (canceled)

37. A method for creating a shunt, comprising:
positioning one or more loops of a snare catheter within a right pulmonary artery;
positioning a crossing catheter and a puncturing guidewire within a superior vena cava such that their distal ends are positioned near the one or more loops of the snare catheter;
advancing the puncturing guidewire out of the superior vena cava and into the right pulmonary artery; and,
advancing the crossing catheter from the superior vena cava to the right pulmonary artery.

38-47. (canceled)
